(19) United States
(12) Patent Application Publication
(10) Pub. No.: US 2008/0071238 A1
(43) Pub. Date: Mar. 20, 2008

(54) Absorbent articles including an improved odor control system

(76) Inventors: Giancarlo Sierri, Montefino (IT); Mariangela Caputi, Molfetta (BA) (IT); Remo Bellucci, Spoltore (PE) (IT); Luigia D'Ercole, Montesilvano (IT)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/881,586

(22) Filed: Jul. 27, 2007

(30) Foreign Application Priority Data
Aug. 9, 2006 (EP) 06016652.7

(51) Int. Cl. A61F 13/15

(52) U.S. Cl. 604/359; 604/378

(57) ABSTRACT

Absorbent articles with an odor control system. The odor control system includes at least two classes of odor control materials, wherein a first class comprises at least a material having low volatility, and a second class comprises at least a material having high volatility. The odor control system is provided in the absorbent article in a selected position and amount to provide a prolonged odor control action.

### FIELD OF THE INVENTION

The present invention relates to absorbent articles which are provided with an odor control system. The odor control system comprises classes of odor control materials, wherein one class comprises materials with low volatility and a second class comprises materials with high volatility. The classes of odor control materials may be selected to provide a synergistic effect in terms of malodor reduction, and the odor control system is positioned in the absorbent article in order to provide an improved odor control action over a longer time.

### BACKGROUND OF THE INVENTION

Absorbent articles of personal hygiene are known in the art. Typical examples include sanitary napkins, panty liners, adult incontinence articles, infant diapers, paper towels, bath tissue and facial tissue. Such articles are often used to absorb and retain bodily fluids and other exudates excreted by the human body. Articles for personal feminine hygiene such as sanitary napkins, panty liners and articles for light adult incontinence are often used by applying them to the crotch portion of an undergarment by means of adhesive means. The adhesive may be provided onto the backsheet garment side and protected by a release layer which is removed before use in order to expose the adhesive. Often, such fluids and exudates are perceived as malodorous and offensive. Therefore, methods and materials for controlling and reducing malodors in absorbent articles have been developed. However, due to the nature of action and the materials chosen only a limited variety of malodorous compounds can be counteracted.

Therefore it is desirable to provide absorbent articles having an odor control system, which acts against a wide variety of malodors in a holistic manner. It is also desirable to provide an absorbent article with an odor control system which is capable of ensuring a sustained action over time, i.e. starting from the moment the article is taken from its package, and continuing during its use. Desirably the odor control system comprises at least one class of odor control materials having low volatility, and at least another class of odor control materials having high volatility. In articles intended to be applied to the crotch portion of an undergarment by means of an adhesive layer, it may be desirable that the odor control materials are prevented from emanating until the release layer is removed from the article, typically by detaching it from the panty fastening adhesive layer provided onto the absorbent article backsheet.

It is also desirable to provide an absorbent article having an odor control system where the at least one class of materials comprising low volatility odor control materials reduces malodors by acting on malodors or a malodorous substance in the article and the at least other class of materials comprising high volatility odor control materials acts on certain nose receptors, typically to help reduce the perception of malodor, or to provide a pleasant odor.

### SUMMARY OF THE INVENTION

The present invention addresses the above need by providing an absorbent article comprising a liquid permeable topsheet, a backsheet, and an absorbent core comprised between the topsheet and the backsheet, each of the topsheet, backsheet and absorbent core having a body facing side and a garment facing side; the garment facing side of the core also has a core surface area. The absorbent article comprises an odor control system comprising first and second classes of odor control materials, wherein the first class comprises at least a material having a Kovat Index (KI) higher than about 1500, and the second class comprises at least a material having a Kovat Index of about 1500 or less. At least the second class of odor control materials of the odor control system is provided onto the garment facing side of the absorbent core, adjacent to the body facing side of the backsheet, on a surface area of at least about 900 mm².

### DETAILED DESCRIPTION OF THE INVENTION

The term “absorbent article” is used herein in a very broad sense including any article able to receive and/or absorb and/or contain and/or retain fluids and/or exudates, especially bodily fluids/bodily exudates. Exemplary absorbent articles in the context of the present invention are disposable absorbent articles.

The term “disposable” is used herein to describe articles, which are not intended to be laundered or otherwise restored or reused as an article (i.e. they are intended to be discarded after a single use and preferably to be recycled, composted or otherwise disposed of in an environmentally compatible manner). Typical disposable absorbent articles according to the present invention can be absorbent articles for feminine hygiene such as sanitary napkins, panty liners, light incontinence products, or the like. Absorbent articles according to the present invention can also encompass diapers, surgical and wound dressings and perspiration pads, incontinence pads.

The term “use”, as used herein, refers to the period of time that starts when the absorbent article is actually put in contact with the anatomy of a wearer.

By “body fluid” it is meant herein any fluid produced by human body including, but not limited to, perspiration, urine, menstrual fluids, vaginal secretions and the like.

The present invention relates to an absorbent article comprising an odor control system, which reduces the malodor generated by a broad range of odorous materials typically occurring in or resulting from the degradation of body fluids and/or materials making up the absorbent article. The odor control system herein includes at least two classes of odor control materials; a first class comprises at least an odor control material having a low volatility, and a second class comprises at least an odor control material having a high volatility. In an embodiment of the present invention, one class of odor control materials, typically the first class above, counteracts the malodors of the exudates or other malodorous material (also referred to herein as acting “internally”), whereas typically the second class counteracts such malodors by affecting the receptors in the nose.

Absorbent Article

The absorbent article of the present invention can be any kind of absorbent article for personal hygiene known in the art including absorbent articles for feminine hygiene. Such articles typically comprise a liquid permeable topsheet, a backsheet, and an absorbent core therebetween. Each of these elements, as well as any other optional layer present in the absorbent article, has a body facing side or wearer facing side, and a garment facing side or outer facing side, which correspond to the side facing respectively the body and the garment of the wearer during use of the product.

As it is known in the art, topsheets may be manufactured from a wide range of materials which include, but are not limited to, woven and nonwoven materials; polymeric materials such as apertured formed thermoplastic films, apertured plastic films, and hydroformed thermoplastic films; porous foams; reticulated foams; reticulated thermoplastic films; and thermoplastic scrims. A topsheet is typically a specific separate element in the absorbent article of the present invention, comprising one or more layers; however, in an absorbent article according to the present invention the topsheet is meant to correspond to the layer or element which in use goes in direct contact with the user's body, for example, the topsheet can be the topmost layer of the absorbent core, being substantially part of the core itself.

The absorbent core can be any absorbent member which is generally compressible, conformable, non-irritating to the wearer's skin, and capable of absorbing and retaining liquids such as urine and other certain body exudates. The absorbent core may be manufactured in a wide variety of sizes and shapes and from a wide variety of liquid-absorbent materials such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials. The configuration and construction of the absorbent core may vary. For example, the absorbent core may have varying caliper zones, a hydrophilic gradient, a superabsorbent gradient, or lower average density and lower average basis weight acquisition zones; or may include one or more layers or structures.

The absorbent core may include other optional components. One such optional component is a core wrap, i.e., a material which either partially or totally surrounds the core. Suitable core wrap materials include, but are not limited to, cellulose, nonwoven materials, perforated films and combinations thereof.

The backsheet may be impervious to liquids and can be manufactured from a thin plastic film. The backsheet may permit vapors to escape from the disposable absorbent article; for example, a microporous polyethylene film can be used for the backsheet. One suitable material for the backsheet of the absorbent article of the present invention can be a liquid impervious thermoplastic film having a thickness of from about 0.012 mm to about 0.051 mm, for example including polyethylene or polypropylene. The backsheet can have a basis weight of from about 5 g/m² to about 35 g/m². However, it should be noted that other materials may be used as the backsheet. The backsheet is typically positioned adjacent the outer-facing side of the absorbent core, and can be joined thereto by any suitable attachment means known in the art. For example, the backsheet may be secured to the absorbent core by a uniform continuous layer of adhesive, a patterned layer of adhesive, or an array of separate lines, spirals, or spots of adhesive.

When the absorbent article of the present invention is an article for feminine hygiene like a sanitary napkin, a panty liner, or an article for light incontinence, it is typically used by being adhered to the crotch portion of an undergarment during use by attachment means, typically a layer of pressure sensitive adhesive that is usually referred to as the panty fastening adhesive, provided onto the garment facing side of the backsheet. Before use, the panty fastening adhesive may be protected by a release layer releasably adhered thereto, which is removed by the user to expose the adhesive when the article is to be applied to the undergarment. As it is known in the art, the release layer may be for example a sheet of siliconized paper, or a wrapper sheet, typically made of a polymeric film, which may also provide a releasable unitary wrapper for the article.

According to the present invention, the absorbent article further comprises an odor control system comprising first and second classes of odor control materials. The first class of odor control materials comprises at least one material having a low volatility, and the second class of odor control materials comprises at least one material having a high volatility. In the context of the present invention, the volatility is expressed in terms of Kovat Index (KI) which, as it is known in the art, is defined as the selective retention of perfume raw materials (PRMs) onto chromatographic columns. The values of the Kovat Index are obtained with a chromatographic column DB-5, 30 m, 0.25 mm, 1.00 μm, available from Agilent Technologies Inc. (formerly J&W Scientific), operating under the following conditions: 50-300° C., 4° C./min, 12.0 psi, constant flow; or equivalent equipment working under same or equivalent conditions, as can be readily determined by the man skilled in the art.

Although primarily defined for perfume raw materials (PRMs), a Kovat Index can be measured generally for liquid and also gaseous materials which are not necessarily perfume raw materials (PRM), and which can be comprised in the first and second class of odor control materials in the odor control system of the absorbent article of the present invention, as will be explained hereinafter. As known, a material's, or more specifically a PRM's polarity, molecular weight, vapor pressure, boiling point, and the stationary phase property determine the extent of retention, and hence of the Kovat Index. According to the present invention, the at least one low volatility material in the first class of odor control materials has a Kovat Index higher than 1500, or between 1550 and 1900; and the at least one high volatility material in the second class of odor control materials has a Kovat Index of 1500 or less, or between 900 and 1500, or also between 1000 and 1400.

At least the second class of the odor control system of the absorbent article of the present invention is disposed on the garment facing side of the absorbent core, adjacent to the body facing side of the backsheet, on a surface area of at least 900 mm², or of 900 mm² to 2000 mm², or also of 900 mm² to 1300 mm². In certain embodiments, at least the second class of odor control materials is disposed on the garment facing side of the absorbent core, adjacent to the body facing side of the backsheet, on a surface area such that the ratio of this surface area to the core surface area is at least 0.08, or between 0.08 and 0.2, or also between 0.08 and 0.1, wherein the core surface area is defined as the surface area of the garment facing side of the absorbent core element. By “adjacent”, it is meant that the odor control system, applied onto the garment facing side of the absorbent core, is located in the finished product substantially towards the body facing side of the backsheet. In an embodiment of the present invention, the odor control system is in direct contact with the body facing side of the backsheet, i.e., without any further layer interposed between the absorbent core and the backsheet. However, “adjacent” also means that an intermediate layer can be interposed between the garment facing side of the core, where the odor control system is applied, and the body facing side of the backsheet, for example a core wrap, or a tissue layer, e.g. a distribution layer. An optional adhesive means between the core and the backsheet is not typically considered as a further layer in the context of the present invention.

The second class of odor control materials can be applied according to any suitable method to the garment facing side of the absorbent core of the absorbent article of the present invention. Typical materials of the second class of odor control materials are liquid at room temperature, as will become apparent in more details further on, and therefore can be applied with known means to the garment facing side of the absorbent core, for example in drops, in any desired regular or irregular pattern. In one exemplary embodiment of the present invention, the second class of odor control materials of the odor control system is provided in one or more longitudinal stripes, for example in two longitudinal stripes parallel to each other and to a longitudinal symmetry axis of the absorbent article, and running symmetrically along substantially the entire core length at both sides of this longitudinal axis.

According to an embodiment of the present invention, at least the second class of odor control materials of the odor control system is provided onto the garment facing side of the core in a basis weight of about 5 g/m² to about 100 g/m², or of about 10 g/m² to about 80 g/m², or also of about 20 g/m² to about 60 g/m².

According to an embodiment of the present invention, the first class of odor control materials typically comprises materials which act on malodors of the exudates or fluids, or other malodorous substances which are present in the absorbent article during its use, while the second class of odor control materials typically comprises substances which act on the user's nose receptors.

Without being bound to any theory, it is believed that an odor control system according to the present invention and comprising a first class of odor control materials typically having a low volatility, and a second class of odor control materials typically having a high volatility, when applied on the garment facing side of the core on the selected surface area and optionally in the selected basis weight, being adjacent to the body facing side of the backsheet, or in an embodiment in direct contact thereto, i.e. with no intermediate layer interposed, such as a core wrapper, causes the more volatile material or materials of the odor control system, typically that or those in the second class of odor control materials, to diffuse through the backsheet layer itself, which is typically a polymeric film, usually a polyethylene film having a thickness in the range of 10-40 μm, e.g. about 20 μm. The more volatile material or materials are however may be prevented from escaping outside of the absorbent article at least in part by the resistance to diffusion opposed by the backsheet material itself, and particularly by the layer of panty fastening adhesive provided on the garment facing side of the backsheet of the typical absorbent articles of the present invention, namely feminine hygiene articles such as sanitary napkins, panty liners, and articles for light adult incontinence, and by the release layer applied thereto. At the same time, the more volatile material or materials of the odor control system have a minor tendency to escape from the topsheet of the absorbent article, owing to the greater thickness of the absorbent article layers and materials overlying the odor control system in the direction of the body facing side of the article. When the user takes the article from its packaging, typically exposes the panty fastening adhesive by removing the release layer in order to apply the article onto the panty, and thereby “freeing” the volatile odor control material or materials entrapped by the panty fastening adhesive and the release layer. The more volatile odor control material or materials therefore may act immediately after the article is readied for use by the user, by typically affecting the user's nose receptors even before the article is actually worn. The more volatile odor control material or materials, together with the less volatile odor control material or materials in the odor control system of the absorbent article of the present invention will also continue to perform their action, respectively towards the user's nose receptors and towards malodors of the exudates or other malodorous material in the absorbent article itself in use. The absorbent article of the present invention, therefore, is capable of providing an improved and prolonged two-step odor control action, owing to the composition of the odor control system and to its particular positioning within the absorbent article, as described above.

Odor Control Materials Acting on the Malodors or Malodorous Substance

The first class of odor control materials in the odor control system of the absorbent article of the present invention comprises at least a material having a Kovat Index higher than about 1500, or between about 1550 and about 1900, hence with a low volatility. The materials of the first class of odor control materials can be selected among known materials which counteract malodors.

There are many materials known in the art for counteracting malodors in absorbent articles. Examples can be found in the references cited herein before. Typical substances are zeolites, starch, activated carbon, cyclodextrine, chitin or chitosan and esters. In general, as it is readily apparent to the man skilled in the art, solid materials such as those mentioned above do not actually have a Kovat Index, which can be only measured for liquid or also gaseous materials. However, solid materials can be said to be substantially non volatile, and in the context of the present invention can be considered to practically satisfy the prescribed condition for the Kovat Index of the at least one low volatility material in the first class of odor control materials in the odor control system according to the present invention.

These actives can reduce the malodor unpleasantness according to different mechanisms, e.g. they can reduce the amount of malodorous molecules through absorption/adsorption mechanisms and/or can react with the malodorous molecules transforming them into low volatile/non-odorous ones and/or can suppress malodorous molecules volatility and/or can prevent the malodor generation by inhibiting degradative processes caused by micro-organisms metabolic activity.

For the odor control system of an embodiment of the present invention a specific selection of such materials may be desired. It has been found that silica gel, aldehydes and mesoporous zeolites are particularly useful.

Silica gel is a porous, amorphous form of silica (SiO₂). It is composed of a vast network of interconnected microscopic pores. As opposed to zeolites, silica gels have larger pores with a wide range of diameters typically between about 5 Å and about 3000 Å. Silica gel, which has proven particularly useful in the odor control system herein, is the narrow silica gel with a pH of less than 7. Indeed it was discovered that this type of silica gel is effective in reducing the malodor level according to two different types of odor control mechanisms: absorption/adsorption of malodorous molecules on silica surface and neutralization of aminic components. The latter are a main source of malodor especially in feminine hygienic products. Silica gels with a pH of less than 6 may be desirable for certain embodiments.

It is possible to adjust the silica gel pore size range in the manufacturing process: Silica gels synthesized with an average pore size of about 10-50 Å are known as “narrow” pore silica gels; silica gels with an average pore size of about 110 Å and beyond are called “wide” pore silica gels. Silica gels with wide pore are generally more expensive than narrow silica gel. In certain embodiments, the silica gel used herein is the narrow silica gel with average pore size of from about 20 to about 40 Å, or in some embodiments about 30 Å.

One suitable silica gel for the odor control system of the absorbent article of the present invention herein has a total surface area higher than about 500 m²/g. The total surface area can be established by using the BET (Brunauer-Emmett-Teller) test. This test is based on the adsorption of nitrogen gas at 77° K onto the surfaces of the silica gel particles, i.e. also their internal cavities. The adsorbed volume of nitrogen is then established by comparing the nitrogen pressure before and after the adsorption. Exemplary suitable silica gel materials include silica gel code 122 and 123 available from Grace, Columbia, Md., USA.

It has further been found that narrow silica gel or zeolites, especially mesoporous zeolites, can be used to stabilize the volatile odor control materials acting externally in the absorbent article. Mesoporous zeolites are those zeolites with pore size from about 20 to about 500 Å. As indicated above, silica gel can absorb volatile substances and thus reduces its migration out of the absorbent article. Advantageously the shelf life of the absorbent articles is significantly prolonged thereby.

Without being bound by theory, it is assumed that narrow silica gel can adsorb the most volatile and active compounds due to its porosity, particularly by creation of hydrogen bonding, and easily release active compounds during usage of product, i.e. due to presence of water that competes with the absorbed molecules in the formation of hydrogen bonds with the silica gel surface.

The stabilization effect has been proven by running a Thermogravimetric analysis (TGA) on samples of narrow silica gel treated with 10 weight-% of menthyl acetate. Specifically, the method is based on evaluation of weight loss over time at specific temperature (40° C.). The samples were kept at 40° C. for 180 minutes. In order to have a basis for comparison the TGA analysis was performed on a sample of pure menthyl acetate and a sample of pure silica gel as references together with a sample of menthyl acetate (10%)+silica gel (ex Grace, coded 123). The results are listed in table 1 and illustrate that the sample with silica gel and MA has a significant lower weight loss than the samples of menthyl acetate alone.

TABLE 1

| | | Weight Loss |
|---|---|---|
| Data point | Sample composition | (%) |
| 1 | Menthyl acetate | ˜100% |
| 2 | Silica gel + Menthyl acetate | 3 |
| 3 | Silica gel | 3 |

One component of body fluid malodor is ammonia. For example ammonia is present in high amounts in products used for urine absorption due to degradation of urea. Ammonia and its derivatives can react with aldehyde to form imines (according to the so-called Schiff base reaction).

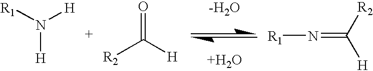

This reaction is catalyzed by enzymes and/or by a slightly acidic pH 4 to 5. The moderate acid requirement is necessary to allow protonation of the hydroxyl intermediate to allow water to leave.

Unfortunately, most aldehydes capable of imine reaction have an unpleasant and/or too intense odor that can be disturbing to human nose and/or they are very volatile and so not stable on the product. Therefore, it is desirable to select suitable materials for controlling malodor. Examples of suitable aldehydes for controlling malodor are those aldehydes that are able to react with aminic compounds according to Schiff base reaction and have not unpleasant odor. Suitable aldehydes include hexyl cinnamic aldehyde, alpha-amylcinnamic aldehyde, p-anisaldehyde, benzaldehyde, cinnamic aldehyde, cuminic aldehyde, decanal, p-t-butyl-alpha-methyldihydrocinnamaldehyde, 4-hydroxy-3-methoxycinnamaldehyde, 2-phenyl-3-(2-furyl)prop-2-enal, vanillin isobutyrate, ethyl vanillin acetate, vanillin acetate, cyclamen aldehyde, heptanal, lauryl aldehyde, nonanal, octanal, phenylacetaldehyde, phenyl propyl aldehyde, vanillin, salycil aldehyde, cytral, 2,4-dihydroxy-3-methylbenzaldehyde, 2-hydroxy-4-methylbenzaldehyde, 5-methyl salicylic aldehydes, 4-nitrobenzaldehyde, o-nitrobenzaldehyde, 5-ethyl-2-thiophenecarbaldehyde, 5-methyl-2-thiophenecarboxaldehyde, 2-thiophenecarbaldehyde, asaronaldehyde, 5-(hydroxymethyl)-2-furaldehyde, 2-benzofurancarboxaldehyde, 2,3,4-trimethoxybenzaldehyde, protocatechualdehyde, heliotropine, 4-ethoxy-3-methoxy benzaldehyde, 3,4,5-trimethoxybenzaldehyde, 3-hydroxybenzaldehyde, o-methoxycinnamaldehyde, 3,5-dimethoxy-4-hydroxycinnamaldehyde, 2,8-dithianon-4-3n-4-carboxaldehyde, sorbinaldehyde, 2,4-heptadienal, 2,4-decadienal, 2,4-nonadienal, 2,4-nonadienal, (E,E)-,2,4-octadien-1-al, 2,4-octadienal, 2,4-dodecadienal, 2,4-undecadienal, 2,4-tridecadien-1-al, 2-trans-4-cis-7-cis-tridecatrienal, piperonylidene propionaldehyde, 2-methyl-3-(2-furyl)acrolein, 2,4-pentadienal, 2-furfurylidene butyraldehyde, 3-(2-furyl)acrolein, pyruvaldehyde, ethanedial and mixtures thereof. Particularly suitable aldehydes are hexyl cinnamic aldehyde, alpha-amylcinnamic aldehyde, decanal, 4-hydroxy-3-methoxycinnamaldehyde, 3,5-dimethoxy-4-hydroxycinnamaldehyde, 2-phenyl-3-(2-furyl)prop-2-enal, ethyl vanillin acetate, vanillin isobutyrate, vanillin acetate, asaronaldehyde. Some of the most desirable aldehydes for application herein are hexyl cinnamic aldehyde, alpha-amylcinnamaldehyde, 4-hydroxy-3-methoxycinnamaldehyde, and decanal.

Odor Control Materials Acting on Nose Receptors

The second class of odor control materials in the odor control system of the absorbent article of the present invention comprises at least a material having a Kovat Index of about 1550 or less, or between about 900 and about 1500, or also between about 1000 and about 1400, hence with a high volatility. In an embodiment of the present invention, all odor control materials of the second class have the prescribed low volatility expressed in terms of the Kovat Index. The materials of the second class of odor control materials can be selected among known materials which act on the nose receptors of the user, e.g. can be perfumes or fragrances, which emanate a pleasant scent.

The second class of odor control materials in the odor control systems hence counteracts odors externally, outside the absorbent articles. According to an embodiment of the present invention, suitable materials of the second class of odor control materials are those listed hereinafter which inhibit the receptors of the nose, hereinafter called “nose blocking”. When used, these materials may significantly reduce the capability for the nose to detect the malodors. The action on the nose receptors, preferably the nose blocking with the selected materials according to this embodiment of the present invention, is possible due to the volatile nature of the materials selected, represented by the selected Kovat Index, which are evaporating out of the absorbent article according to the mechanism described above, and are then inhaled into the nose of an individual generally within somewhat close range of the article, e.g. within about 0 to 10 meters of the article (although this should in no way be intended to limit the scope of the invention) by normal breathing. The blocking of the nose receptors is, of course, only temporary.

Suitable nose blocking materials include menthol, menthyl acetate, 3-buten-2-one,3-methyl-4-(2,6,6-trimethyl-2-cyclohexen-1-yl)-, 4-(2,6,6-trimethylcyclohen-1-en-1-yl)but-3-en-2-one, 3-buten-2-one,4-(2,6,6-trimethyl-2-cyclohexen-1-yl)-, (E)-, menthyl lactate, isomenthyl acetate, isomenthyl propionate, isomenthyl isobutyrate, isomenthyl propionate, isomenthyl butyrate, camphor and p-menthane. These materials also include their isomeric forms, diastereomers and enantiomers. Advantageously, in general, the above materials have only a very slight inherent odor but show a high degree of nose receptor blocking.

According to an embodiment of the present invention, an absorbent article comprising topsheet, backsheet and absorbent core can further comprise an odor control system comprising a first class of odor control materials selected from the group consisting of silica gel having a pH of less than 7, mesoporous zeolites having pores sizes of from about 20 to about 500 Å and mixtures thereof, and aldehydes in turn selected from the group as described above, and a second class of odor control materials selected from the group consisting of nose blocking materials as described above, wherein the at least second class of odor control materials of the odor control system is provided onto the garment facing side of the absorbent core, being adjacent to the body facing side of the backsheet, on a surface area of at least about 900 mm², or of about 900 mm² to about 2000 mm², or also of about 900 mm² to about 1300 mm². In certain embodiments, the at least second class of odor control materials is disposed on the garment facing side of the absorbent core, being adjacent to the body facing side of the backsheet, on a surface area such that the ratio of said surface area to said core surface area is at least about 0.08, or between about 0.08 and about 0.2, or also between about 0.08 and about 0.1. According to the above embodiment, any combinations with all other additional or alternative features described herein are also comprised.

According to an alternative embodiment of the present invention, the high volatility materials of the second class of odor control materials can be selected among materials having a pleasant odor. According to the law of Dalton, due to their high partial pressure in the headspace of the absorbent article, and also owing to their application in selected position, areas and basis weights as explained before, such highly volatile components are believed to reduce the molar fraction of the malodorous compounds having a lower partial pressure, and provide a pleasant odor to the user. Suitable highly volatile components include materials that have a KI (Kovat Index) below about 1500.

Suitable highly volatile components that act according to this mechanism include e.g. limonene, eucalyptol, cresol, linalool, tetra-hydrolinalool, myrcenol, tetra hydromyrcenol, di-hydromyrcenol, myrcene, cytronellol, cytronellyil derivatives, geraniol, geranyl derivatives, linalyl acetate, mugetanol, eugenol, jasmal, terpineol, pinanol, cedrene, damascone, beta pinene, cineole and its derivatives, nonadienol, ethylhexanal, octanol acetate, methyl furfural, terpinene, thujene, amylacetate, benzylacetate, camphene, citronellal, di-hydrocumarin, di-hydromyrcenyl acetate, geraniol, geranial, isoamylacetate, ethyl, and/or triethyl acetate, para-cresol and para-cymene.

According to a further embodiment of the present invention, the above high volatility materials having a pleasant odor are included in the second class of odor control materials of the odor control system in the absorbent article of the present invention in addition to the “nose blocking” materials described above.

Optional Further Components

a) Hydrogel Forming Absorbent Polymers

The absorbent article of the present invention can also comprise, in addition to the odor, control system, a hydrogel forming absorbent polymer. Hydrogel forming absorbent polymers useful in the present invention include a variety of water-insoluble, but water-swellable polymers capable of absorbing large quantities of fluids, and are also commonly referred to in the art as absorbent gelling materials, or superabsorbent materials. Absorbent gelling materials, as it is known in the art, are widely used to provide absorbent articles with improved absorbent capacity, being typically comprised in absorbent structures, alone or in combination with fibrous absorbent material such as for example pulp, airfelt, tissue or nonwoven layers. Absorbent gelling materials are also used in combination with known odor control materials, as they can provide additional benefit in odor control capability together with their liquid absorption capacity.

Any absorbent gelling materials which are known in the art can be comprised in the absorbent article of the present invention, in addition to the odor control system. Suitable absorbent gelling materials can include hydrolyzed acrylonitrile grafted starch, acrylic acid grafted starch, polyacrylates, maleic anhydride-based copolymers and combinations thereof. Particularly suitable absorbent gelling materials are the polyacrylates and acrylic acid grafted starch.

The absorbent gelling materials herein before described are typically used in the form of discrete particles. Such absorbent gelling materials can be of any desired shape, e.g., spherical or semi-spherical, cubic, rod-like polyhedral, etc. Shapes having a large greatest dimension/smallest dimension ratio, like needles and flakes, are also contemplated for use herein. Agglomerates of absorbent gelling material particles may also be used.

The size of the absorbent gelling material particles may vary over a wide range. For example, particle sizes comprised between about 150μ and about 800μ are commonly used in the art, although smaller or greater particle sizes can be also used. “Particle Size” as used herein means the weighted average of the smallest dimension of the individual particles. The absorbent gelling materials are incorporated into the absorbent article of the present invention according to known means, typically within the absorbent core.

b) Solvents

Further additional ingredients include solvents as carriers for incorporating the odor control materials into the absorbent article. Suitable solvents are e.g. benzyl-benzoate, isopropyl myristate, methyl abietate, ethanol, isopropanol, diethylene glycol monoethyl ether, glycerol, propylene glycol, 1,2-butylene glycol, dipropylene glycol, 2-methyl-2,4-pentanediol, diethyl phthalate, trietyl citrate, diethyl sebacate.

The Odor Control System

It has been found that the combination of certain, separately known odor control materials results in a synergistic effect in terms of odor reduction. Specifically, in an embodiment of the present invention a synergistic effect in terms of malodor reduction is observed when combining odor control materials acting on the malodors or malodorous substance itself and odor control materials acting on nose receptors, as described above. In fact, an odor control system combining actives of the aforementioned two classes may reduce the malodors more efficiently than the mathematical combination of each material acting individually.

Without wishing to be bound by theory this is believed to be a result of the neutralization of the malodorous molecules in the absorbent article by the odor control materials acting internally, which reduces the concentration of these molecules in the head space and thus, in the air. Due to this, and in combination with the selected positioning in the absorbent article of the present invention, as explained above, the odor control materials acting externally onto the nose receptors can exploit their maximum activity.

When the odor control system of the present invention is used in absorbent articles the individual odor control materials can be employed at variable amounts. When considering the specific suitable odor control materials of the first class, namely odor control materials acting internally, for silica gel an amount of from 5 g/m² to 300 g/m², or from 20 g/m² to 100 g/m² has proven useful. For aldehydes, an amount of from 0.05 g/m² to 20 g/m², or from 0.5 g/m² to 5 g/m² has proven useful. An example of a suitable odor control material of the second class, namely a material acting on nose receptors, can include menthyl acetate in the range from 0.05 g/m² to 20 g/m², or from 0.5 g/m² to 5 g/m².

The odor control system may comprise the first class of odor control materials, for example typically acting on the malodors or malodorous substance, in relation to the second class of odor control materials, for example typically acting on the nose receptors, at a ratio of from about 50:1 to about 1:50 by weight, or from about 30:1 to about 1:30 by weight or also from about 1:15 to about 15:1 by weight. In one embodiment, the odor control system can comprise silica gel and menthyl acetate at a ratio of about 15:1 by weight. In an alternative embodiment the odor control system can comprise alpha-amylcinnamaldehyde and menthyl acetate at a ratio of about 1:1 by weight.

Exemplary Absorbent Article

The absorbent article being provided with the odor control system herein can be any kind of absorbent article of personal hygiene known in the art. Particularly suitable are absorbent articles for personal feminine hygiene such as sanitary napkins, pantiliners and articles for light adult incontinence. The odor control system of the present invention can be present in any part of the absorbent article, provided at least the second class of odor control material is applied onto the garment facing side of the absorbent core in the prescribed surface area. According to the present invention the odor control system can either be present in the absorbent article as an intimate mixture of the at least two classes of odor control materials or with both classes of odor control materials being separate from each other. For example, an embodiment of the present invention can have the first class of odor control materials (acting on the malodors or malodorous substance) placed on the wearer-facing side of the absorbent core, or alternatively within the structure of the absorbent core, while the second class of odor control materials (acting on nose receptors) can be placed on the garment-facing side of the absorbent core. Taking into account the stabilization described herein before, one execution of this embodiment can be an absorbent article being provided with silica gel on the wearer-facing side of the absorbent core and with menthyl acetate on the garment-facing side of the absorbent core. However, other arrangements of the first and second odor control materials are contemplated.

Test Methods and Data

For proving the synergistic effect on odor reduction of an odor control system according to an embodiment of the present invention several test samples have been prepared, which are all being exposed to a 0.1% aqueous solution of ammonia, which serves as a test malodorous substance. The first data point in table 2 serves as a benchmark as it represents the degree of unpleasantness of the ammonia solution without added odor control material. Data points 2-3 in table 2 are illustrating the malodor-reducing activity of two exemplary odor control materials alone, specifically alpha-amylcinnamaldehyde as exemplary aldehyde and menthyl acetate as exemplary odor control material (“nose blocking” type) acting on the nose receptors.

The last data point 4 in table 2 is illustrating the activity of the odor control system of this embodiment of the present invention. For comparability two individual odor control materials were mixed at half their amount compared to data points 2-3. Data point 4 has been obtained by testing a mixture of alpha-amylcinnamaldehyde and menthyl acetate. It is clearly obtainable from data point 4 that the odor reduction performance of the odor control system is significantly better than the one of the individual odor control materials. Thus, synergistic odor control activity has been proven.

TABLE 2

| | | Odor |
|---|---|---|
| Data | | unpleasant- |
| point # | Sample composition | ness (%) |
| 1 | Malodorant solution (10 ml) | 100 |
| 2 | Alpha-Amylcinnamaldehyde (16 mg) + | 64 |
| | Malodorant solution (10 ml) | |
| 3 | Menthyl acetate (16 mg) + Malodorant | 46 |
| | solution (10 ml) | |
| 4 | Alpha-Amylcinnamaldehyde (8 mg) + | 30 |
| | Menthyl acetate (8 mg) + | |
| | Malodorant solution (10 ml) | |

The odor unpleasantness and pleasantness of these samples have been evaluated by a panel of expert graders. In particular five different expert graders have evaluated 4 replicates for each sample. Odor unpleasantness have been evaluated by using a scale from −10 to +0, where −10 indicates the max odor unpleasantness, 0 indicates no odor. Data have been then reported as % relative unpleasantness vs. Reference (malodorant solution). The odor evaluation has been performed in adequate room, at controlled temperature T (25° C.). The room is equipped with appropriate conditioning system allowing continuous exchange of air. The samples were held in numbered metal trays, which were covered with aluminum foil between the actual gradings.

Each dimension for which a value is defined herein is a technical dimension, which, in the context of the present invention is not to be understood literal. Hence, all embodiments having dimensions functionally equivalent to the dimensions stated herein are intended to be covered by the scope of the invention, e.g. a dimension of “40 mm” has to be understood as meaning “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a liquid permeable topsheet, a backsheet, and an absorbent core between said topsheet and said backsheet, each of said topsheet, backsheet and absorbent core having a body facing side and a garment facing side, said garment facing side of said core having a core surface area, said absorbent article comprising an odor control system comprising first and second classes of odor control materials, wherein said first class comprises at least a material having a Kovat Index (KI) higher than about 1500 and said second class comprises at least one material having a Kovat Index of about 1500 or less,
at least said second class of odor control materials of said odor control system disposed on said garment facing side of said absorbent core, and being adjacent to the body facing side of said backsheet on a surface area of at least about 900 mm².

2. An absorbent article according to claim 1, wherein at least said second class of odor control materials of said odor control system is provided in a basis weight from about 5 g/m² to about 100 g/m².

3. An absorbent article according to claim 1, wherein at least said second class of odor control materials of said odor control system is provided onto said garment facing side of said absorbent core in one or more longitudinal stripes.

4. An absorbent article according to claim 1, wherein said first class of odor control materials reduces odor by acting on malodors or a malodorous substance in the absorbent article and said second class of odor control material reduces odor by acting on the user's nose receptors, wherein said first class of odor control materials is selected from the group consisting of silica gel having a pH of less than 7, aldehydes, mesoporous zeolites having pores sizes of from 20 to 500 Å and mixtures thereof, wherein said aldehydes are selected from the group consisting of hexyl cinnamic aldehyde, alpha-amylcinnamic aldehyde, p-anisaldehyde, benzaldehyde, cinnamic aldehyde, cuminic aldehyde, decanal, cyclamen aldehyde, p-t-butyl-alpha-methyldihydrocinnamaldehyde, 4-hydroxy-3-methoxycinnamaldehyde, vanillin isobutyrate, 2-phenyl-3-(2-furyl)prop-2-enal, ethyl vanillin acetate, vanillin acetate, heptanal, lauryl aldehyde, nonanal, octanal, phenylacetaldehyde, phenyl propyl aldehyde, vanillin, salycil aldehyde, cytral, 2,4-dihydroxy-3-methylbenzaldehyde, 2-hydroxy-4-methylbenzaldehyde, 5-methyl salicylic aldehydes, 4-nitrobenzaldehyde, o-nitrobenzaldehyde, 5-ethyl-2-thiophenecarbaldehyde, 5-methyl-2-thiophenecarboxaldehyde, 2-thiophenecarbaldehyde, asaronaldehyde, 5-(hydroxymethyl)-2-furaldehyde, 2-benzofurancarboxaldehyde, 2,3,4-trimethoxybenzaldehyde, protocatechualdehyde, heliotropine, 4-ethoxy-3-methoxy benzaldehyde, 3,4,5-trimethoxybenzaldehyde, 3-hydroxybenzaldehyde, o-methoxycinnamaldehyde, 3,5-dimethoxy-4-hydroxycinnamaldehyde, 2,8-dithianon-4-3n-4-carboxaldehyde, sorbinaldehyde, 2,4-heptadienal, 2,4-decadienal, 2,4-nonadienal, 2,4-nonadienal, (E,E)-, 2,4-octadien-1-al, 2,4-octadienal, 2,4-dodecadienal, 2,4-undecadienal, 2,4-tridecadien-1-al, 2-trans-4-cis-7-cis-tridecatrienal, piperonylidene propionaldehyde, 2-methyl-3-(2-furyl)acrolein, 2,4-pentadienal, 2-furfurylidene butyraldehyde, 3-(2-furyl)acrolein, pyruvaldehyde, ethanedial and mixtures thereof,
and said second class of odor control materials is selected from the group consisting of menthol, menthyl acetate, menthyl lactate, 3-buten-2-one,3-methyl-4-(2,6,6-trimethyl-2-cyclohexen-1-yl)-, 4-(2,6,6-trimethylcyclohen-1-en-1-yl)but-3-en-2-one, 3-buten-2-one,4-(2,6,6-trimethyl-2-cyclohexen-1-yl)-, (E)-isomenthyl acetate, isomenthyl propionate, isomenthyl isobutyrate, isomenthyl propionate, isomenthyl butyrate, camphor, p-menthane and mixtures thereof.

5. The absorbent article of claim 1, wherein each material in said second class of odor control materials has a Kovat Index of about 1500 or less.

6. The absorbent article of claim 1, wherein the odor control system further comprises a highly volatile component selected from the group consisting of limonene, eucalyptol, cresol, linalool, tetra-hydrolinalool, myrcenol, tetra hydromyrcenol, di-hydromyrcenol, myrcene, cytronellol, cytronellyil derivatives, geraniol, geranyl derivatives, linalyl acetate, mugetanol, eugenol, jasmal, terpineol, pinanol, cedrene, damascone, beta pinene, cineole and its derivatives, nonadienol, ethylhexanal, octanol acetate, methyl furfural, terpinene, thujene, amylacetate, benzylacetate, camphene, citronellal, dihydrocumarin, dy hydromyrcenyl acetate, geraniol, geranial, isoamylacetate, ethyl, and/or triethyl acetate, para-cresol, para-cymene, methyl abietate and mixtures thereof.

7. The absorbent article of claim 4, wherein the odor control system comprises narrow silica gel in an amount from about 5 g/m² to about 300 g/m².

8. The absorbent article of claim 4, wherein the odor control system comprises said aldehyde or aldehydes in an amount from about 0.05 g/m² to about 20 g/m².

9. The absorbent article of claim 4, wherein the odor control system comprises hexyl cinnamic aldehyde.

10. The absorbent article of claim 4, wherein the odor control system comprises menthyl acetate in an amount from about 0.05 g/m² to about 20 g/m².

11. The absorbent article of claim 1, wherein the odor control system comprises said first class of odor control materials in relation to said second class of odor control materials at a ratio from about 50:1 to about 1:50 by weight.

12. The absorbent article of claim 11, wherein the odor control system comprises narrow silica gel and menthyl acetate at a ratio of about 15:1 by weight.

13. The absorbent article of claim 11, wherein the odor control system comprises hexyl cinnamic aldehyde and menthyl acetate at a ratio of about 1:1 by weight.

14. The absorbent article of claim 1, wherein said absorbent article is a sanitary napkin, a pantiliner, or a light adult incontinence product, and further comprising an adhesive means on said garment facing side of said backsheet.

15. An absorbent article comprising a liquid permeable topsheet, a backsheet, and an absorbent core between said topsheet and said backsheet, each of said topsheet, backsheet and absorbent core having a body facing side and a garment facing side, said garment facing side of said core having a core surface area, said absorbent article comprising an odor control system comprising first and second classes of odor control materials, wherein said first class comprises at least a material having a Kovat Index (KI) higher than about 1500 and said second class comprises at least one material having a Kovat Index of about 1500 or less,
at least said second class of odor control materials of said odor control system disposed on said garment facing side of said absorbent core and being adjacent to the body facing side of said backsheet on a surface area such that the ratio of said surface area to said core surface area is at least about 0.08.

16. An absorbent article according to claim 15, wherein at least said second class of odor control materials of said odor control system is provided onto said garment facing side of said absorbent core in one or more longitudinal stripes.

17. An absorbent article according to claim 15, wherein said first class of odor control materials reduces odor by acting on malodors or a malodorous substance in the absorbent article and said second class of odor control material reduces odor by acting on the user's nose receptors, wherein said first class of odor control materials is selected from the group consisting of silica gel having a pH of less than 7, aldehydes, mesoporous zeolites having pores sizes of from 20 to 500 Å and mixtures thereof, wherein said aldehydes are selected from the group consisting of hexyl cinnamic aldehyde, alpha-amylcinnamic aldehyde, p-anisaldehyde, benzaldehyde, cinnamic aldehyde, cuminic aldehyde, decanal, cyclamen aldehyde, p-t-butyl-alpha-methyldihydrocinnamaldehyde, 4-hydroxy-3-methoxycinnamaldehyde, vanillin isobutyrate, 2-phenyl-3-(2-furyl)prop-2-enal, ethyl vanillin acetate, vanillin acetate, heptanal, lauryl aldehyde, nonanal, octanal, phenylacetaldehyde, phenyl propyl aldehyde, vanillin, salycil aldehyde, cytral, 2,4-dihydroxy-3-methylbenzaldehyde, 2-hydroxy-4-methylbenzaldehyde, 5-methyl salicylic aldehydes, 4-nitrobenzaldehyde, o-nitrobenzaldehyde, 5-ethyl-2-thiophenecarbaldehyde, 5-methyl-2-thiophenecarboxaldehyde, 2-thiophenecarbaldehyde, asaronaldehyde, 5-(hydroxymethyl)-2-furaldehyde, 2-benzofurancarboxaldehyde, 2,3,4-trimethoxybenzaldehyde, protocatechualdehyde, heliotropine, 4-ethoxy-3-methoxy benzaldehyde, 3,4,5-trimethoxybenzaldehyde, 3-hydroxybenzaldehyde, o-methoxycinnamaldehyde, 3,5-dimethoxy-4-hydroxycinnamaldehyde, 2,8-dithianon-4-3n-4-carboxaldehyde, sorbinaldehyde, 2,4-heptadienal, 2,4-decadienal, 2,4-nonadienal, 2,4-nonadienal, (E,E)-, 2,4-octadien-1-al, 2,4-octadienal, 2,4-dodecadienal, 2,4-undecadienal, 2,4-tridecadien-1-al, 2-trans-4-cis-7-cis-tridecatrienal, piperonylidene propionaldehyde, 2-methyl-3-(2-furyl)acrolein, 2,4-pentadienal, 2-furfurylidene butyraldehyde, 3-(2-furyl)acrolein, pyruvaldehyde, ethanedial and mixtures thereof,
and said second class of odor control materials is selected from the group consisting of menthol, menthyl acetate, menthyl lactate, 3-buten-2-one,3-methyl-4-(2,6,6-trimethyl-2-cyclohexen-1-yl)-, 4-(2,6,6-trimethylcyclohen-1-en-1-yl)but-3-en-2-one, 3-buten-2-one,4-(2,6,6-trimethyl-2-cyclohexen-1-yl)-, (E)-isomenthyl acetate, isomenthyl propionate, isomenthyl isobutyrate, isomenthyl propionate, isomenthyl butyrate, camphor, p-menthane and mixtures thereof.

18. The absorbent article of claim 15, wherein each material in said second class of odor control materials has a Kovat Index of about 1500 or less.

19. The absorbent article of claim 15, wherein the odor control system further comprises a highly volatile component selected from the group consisting of limonene, eucalyptol, cresol, linalool, tetra-hydrolinalool, myrcenol, tetra hydromyrcenol, di-hydromyrcenol, myrcene, cytronellol, cytronellyil derivatives, geraniol, geranyl derivatives, linalyl acetate, mugetanol, eugenol, jasmal, terpineol, pinanol, cedrene, damascone, beta pinene, cineole and its derivatives, nonadienol, ethylhexanal, octanol acetate, methyl furfural, terpinene, thujene, amylacetate, benzylacetate, camphene, citronellal, dihydrocumarin, dy hydromyrcenyl acetate, geraniol, geranial, isoamylacetate, ethyl, and/or triethyl acetate, para-cresol, para-cymene, methyl abietate and mixtures thereof.

20. The absorbent article of claim 17, wherein the odor control system comprises narrow silica gel in an amount from about 5 g/m² to about 300 g/m².

21. The absorbent article of claim 17, wherein the odor control system comprises said aldehyde or aldehydes in an amount from about 0.05 g/m² to about 20 g/m².

22. The absorbent article of claim 17, wherein the odor control system comprises hexyl cinnamic aldehyde.

23. The absorbent article of claim 17, wherein the odor control system comprises menthyl acetate in an amount from about 0.05 g/m² to about 20 g/m².

24. The absorbent article of claim 15, wherein the odor control system comprises said first class of odor control materials in relation to said second class of odor control materials at a ratio from about 50:1 to about 1:50 by weight.

25. The absorbent article of claim 24, wherein the odor control system comprises narrow silica gel and menthyl acetate at a ratio of about 15:1 by weight.

26. The absorbent article of claim 24, wherein the odor control system comprises hexyl cinnamic aldehyde and menthyl acetate at a ratio of about 1:1 by weight.

27. The absorbent article of claim 24, said absorbent article being a sanitary napkin, a pantiliner, or a light adult incontinence product, and further comprising an adhesive means on said garment facing side of said backsheet.
